(19) United States
(12) Patent Application Publication
(10) Pub. No.: US 2017/0202980 A1
(43) Pub. Date: Jul. 20, 2017

(54) METHOD FOR PRODUCING OPTICAL IMAGING CONTRAST AGENT AND OPTICAL IMAGING CONTRAST AGENT

(71) Applicant: CANON KABUSHIKI KAISHA, Tokyo (JP)

(72) Inventor: Keigo Mizusawa, Tokyo (JP)

(21) Appl. No.: 15/408,974

(22) Filed: Jan. 18, 2017

(30) Foreign Application Priority Data
Jan. 20, 2016 (JP) 2016-009306

(51) Int. Cl. A61K 49/00; A61K 49/22

(52) U.S. Cl. CPC A61K 49/0032; A61K 49/0056; A61K 49/221

(57) ABSTRACT

A method for producing an optical imaging contrast agent includes the following process steps: (a) reacting an albumin with a specific organic dye or a salt thereof in an aqueous solution to prepare an aqueous solution containing a combined form of the specific organic dye and the albumin; (b) adjusting the pH of the aqueous solution containing the combined form to 7.2 or less; (c) adding an ester to the aqueous solution having a pH of 7.2 or less and stirring the mixture, thereby preparing a liquid in which an aqueous solution phase and an organic solvent phase containing the ester are separated from each other; and (d) extracting an aqueous solution containing the combined form from the phase-separated liquid.

### BACKGROUND OF THE INVENTION

Field of the Invention

The present disclosure relates to a method for producing an optical imaging contrast agent and an optical imaging contrast agent.

Description of the Related Art

An optical imaging method has been known as a method for non-invasively visualizing in vivo information. In the optical imaging method, in vivo information is imaged by measuring signals such as acoustic waves or fluorescence emitted from a substance (light absorber) in a subject when the substance has absorbed light applied to the subject. Alternatively, indocyanine green (hereinafter abbreviated as ICG) or any other dye that absorbs light having a wavelength in the near infrared region may be administered to a living body, and fluorescence or acoustic waves emitted from the dye in the living body may be measured. ICG and similar organic dyes can be used as an optical imaging contrast agent.

For using an organic dye as a tumor contrast agent, it is advantageous to combine the dye with a macromolecular compound such as a polymer or a protein. This is because organic dyes generally have a low molecular weight and are readily discharged from blood before accumulating in the tumor, whereas a form of the dye combined with a macromolecular compound is unlikely to be discharged and accordingly accumulates in the tumor while circulating in blood, consequently increasing accumulation in the tumor.

The contrast agent also contains the organic dye itself remaining therein even after purification, in addition to the combined form. Since the organic dye itself is readily discharged from blood and does not accumulate in the tumor, as described above, it is desirable that the contrast agent do not contain the organic dye itself. This is because the total content of all forms of the organic dye in the contrast agent is desirably as small as possible in view of burden on the human body. Accordingly, it is desirable that the uncombined form of the organic dye remaining without being combined with the macromolecular compound in a step for producing a combined form be removed after the reaction of the organic dye with the macromolecular compound.

U.S. Pat. No. 6,403,625 discloses a combined form of an organic dye (ICG-ATT) represented by the following formula (1) and bovine serum albumin (hereinafter abbreviated as BSA) that is a protein.

According to this patent disclosure, in order to produce the combined form, ICG-ATT is reacted with BSA in a phosphate aqueous solution of pH 7.5, and the reaction solution is then subjected to gel filtration chromatography using a phosphate aqueous solution of pH 7.5 as the eluent. To the eluent of pH 7.5 thus obtained, ethyl acetate is added, and the mixture is separated into a hydrophobic ethyl acetate phase and a hydrophilic aqueous solution phase by being stirred. ICG-ATT, which is hydrophobic, migrates to the ethyl acetate phase, and the combined form remains in the aqueous solution phase. Finally, the combined form of ICG-ATT and BSA is obtained by collecting the aqueous solution phase.

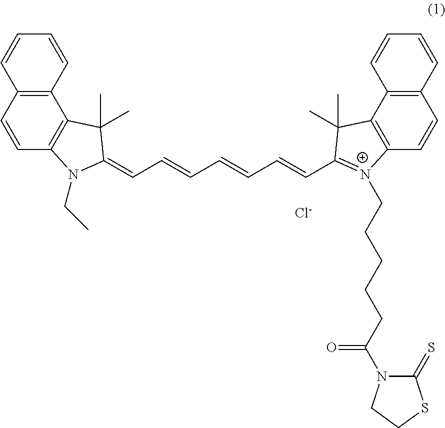 [SMILES: CCN1/C(=C/C=C/C=C/C=C/C2=[N+](CCCCCC(=O)N3CCSC3=S)c3ccc4ccccc4c3C2(C)C)C(C)(C)c2c1ccc1ccccc21 ; [Cl-]]

The organic dye (ICG-ATT) disclosed in the above-cited patent document has a structure in which an ethyl group is bound to the nitrogen of one of the indole rings and is therefore highly hydrophobic and likely to form an aggregate before being combined with the albumin. Thus, the uncombined form of the dye not combined with the albumin is more likely to remain. If a large portion of the dye remains in the aqueous solution after the reaction, it cannot be completely removed by purification using ethyl acetate, as described above, thus remaining also in the contrast agent.

Bioconjugate Chem. Volume 25, Issue 10, pp. 1801-1810 (2014) discloses a combined form of an antibody that is a protein and a dye (ICG-Sulfo-OSu) represented by formula (I-2) that will be described herein later.

In order to produce the combined form in this non-patent document, first, ICG-Sulfo-OSu is reacted with the antibody, followed by gel filtration chromatography using a pH 6.8 solution as the eluent. Then, ethyl acetate is added to the thus obtained eluent, and the mixture is stirred and separated into an ethyl acetate phase and an aqueous solution phase. The aqueous solution phase is collected to obtain the combined form of ICG-Sulfo-OSu and the antibody. The organic dye (ICG-Sulfo-OSu) used in this non-patent document turned hydrophilic by binding an alkyl group substituted by a hydrophilic sulfonic acid group to the nitrogen of the indole ring thereof. The thus obtained combined form is therefore less likely to form an aggregate and can react efficiently with albumin. On the other hand, the uncombined form of the organic dye, remaining without reacting with the albumin, is hydrophilic and is likely to remain in the aqueous solution when the solution is subjected to phase separation by adding ethyl acetate. Consequently, a large portion of the uncombined form of the organic dye remains undesirably in the collected aqueous solution.

### SUMMARY OF THE INVENTION

The present disclosure provides a method for producing an optical imaging contrast agent in which the form of an organic dye not combined with an albumin does not remain much, that is, in which a combined form of the albumin and the organic dye accounts for a high percentage in the contrast agent.

The method for producing an optical imaging contrast agent, according to the present disclosure includes reacting an albumin with an organic compound represented by any one of the following formulas (I) to (III) or a salt thereof in an aqueous solution to prepare an aqueous solution containing a combined form of the organic dye and the albumin with a covalent bond therebetween, adjusting the pH of the aqueous solution containing the combined form to 7.2 or less, adding an ester to the aqueous solution having a pH of 7.2 or less, thereby preparing a liquid in which an organic solvent phase containing the ester and an aqueous solution phase are separated from each other, and extracting an aqueous solution containing the combined form from the phase-separated liquid.

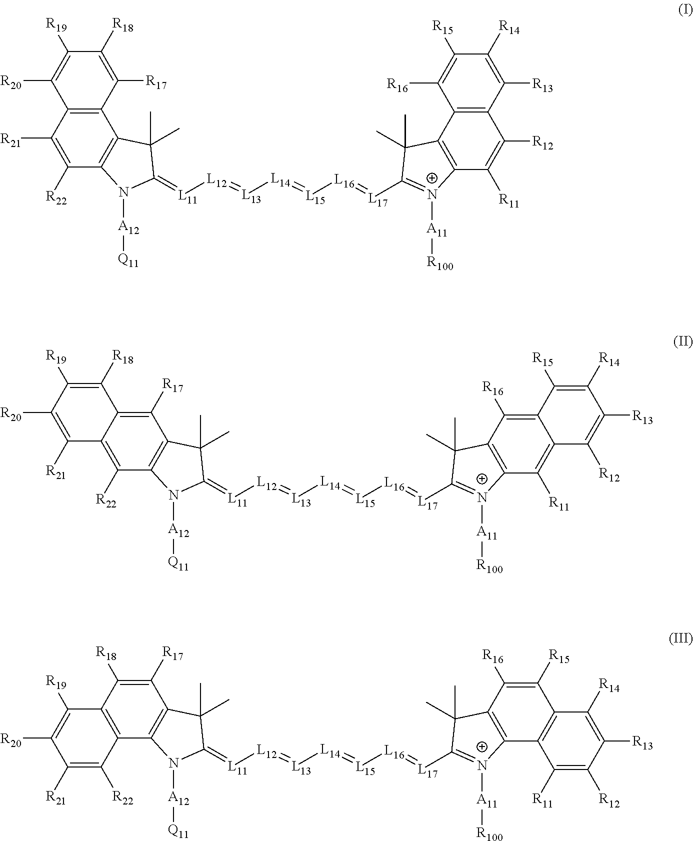

In formulas (I) to (III), R₁₁ to R₂₂ are the same as or different from each other and each represent a chemical species selected from the group consisting of a hydrogen atom, alkyl groups having a carbon number of 1 to 3, and halogen atoms.

L₁₁ to L₁₇ are the same as or different from each other and each represent a methine group that may be substituted by a methyl group or a halogen atom. A₁₁ and A₁₂ are the same as or different from each other and each represent an alkylene group having a carbon number of 1 to 10. Q₁₁ represents one selected from the group consisting of —OH, —CO₂H, —S(═O)₂OH, —P(═O)(OH)₂, and —OP(═O)(OH)₂, and R₁₀₀ represents a group represented by any one of the following formulas (i) to (viii):

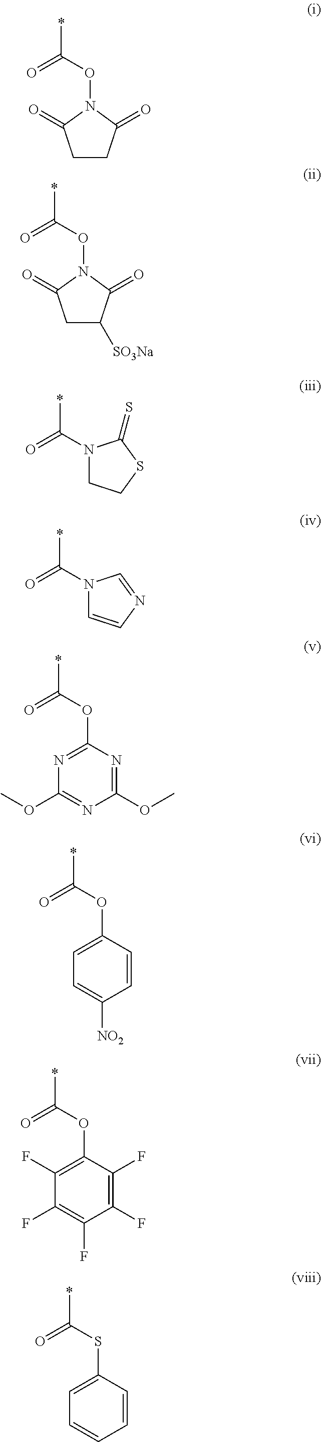

The asterisks in formulas (i) to (viii) each indicate a bonding hand that will form a linkage with A₁₁ in formulas (I) to (III).

An optical imaging contrast agent according to an aspect of the present disclosure includes a combined form of an albumin and an organic dye or a salt thereof with a covalent bond therebetween. The organic dye is represented by any one of the following formulas (I) to (III). The form of the organic dye combined with the albumin accounts for 60% or more of the total moles of all forms of the organic dye in the optical imaging contrast agent.

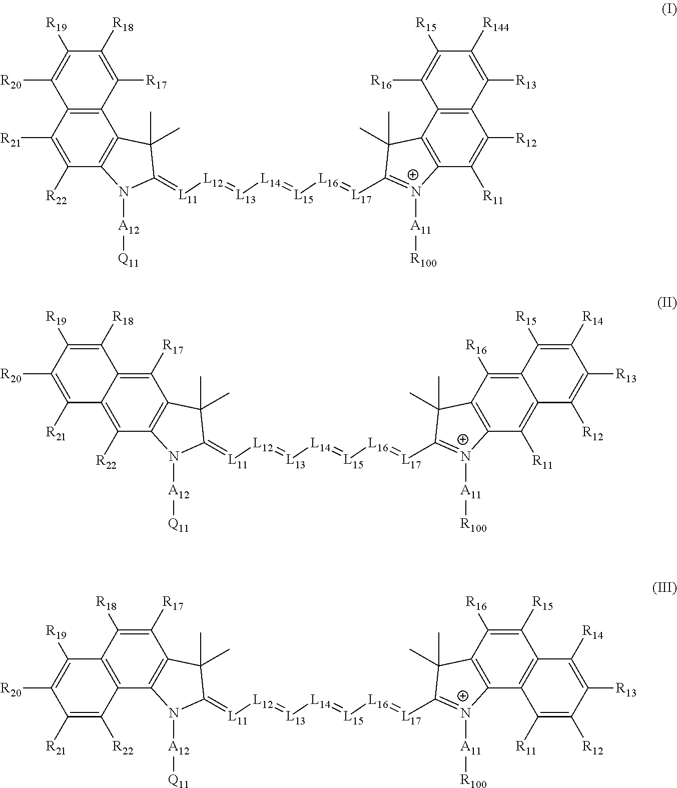

In formulas (I) to (III), R₁₁ to R₂₂ are the same as or different from each other and each represent a chemical species selected from the group consisting of a hydrogen atom, alkyl groups having a carbon number of 1 to 3, and halogen atoms. L₁₁ to L₁₇ are the same as or different from each other and each represent a methine group that may be substituted by a methyl group or a halogen atom. A₁₁ and A₁₂ are the same as or different from each other and each represent an alkylene group having a carbon number of 1 to 10. Q₁₁ represents one selected from the group consisting of —OH, —CO₂H, —S(═O)₂OH, —P(═O)(OH)₂, and —OP(═O)(OH)₂, and R₁₀₀ represents a group represented by any one of the following formulas (i) to (viii):

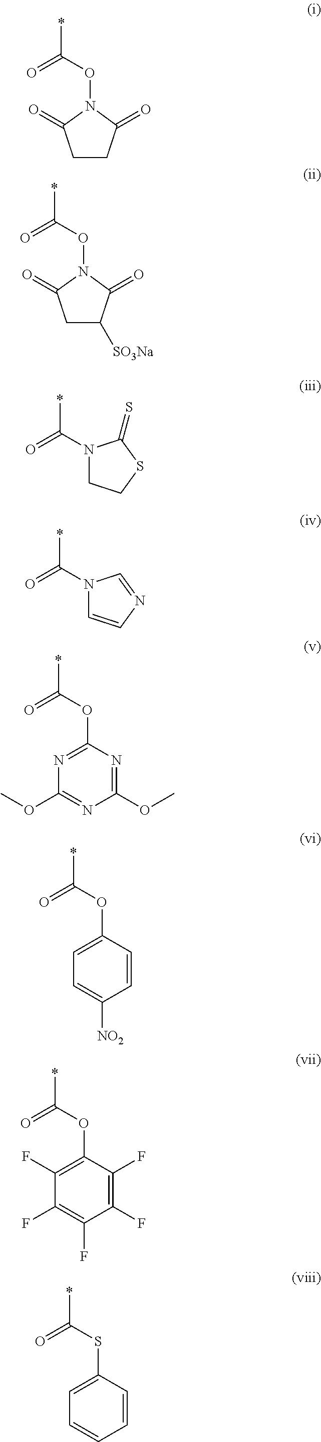

The asterisks * in formulas (i) to (viii) each indicate a bonding hand that will form a linkage with A₁₁ in formulas (I) to (III).

According to still another aspect of the present disclosure, there is provided an optical imaging contrast containing an albumin, and a combined form of an albumin and at least one organic dye or a salt thereof with a covalent bond therebetween, the combined form being represented by any one of the following formulas (IV) to (VI). The form of the organic dye combined with the albumin accounts for 60% or more of the total moles of all forms of the organic dye in the optical imaging contrast agent.

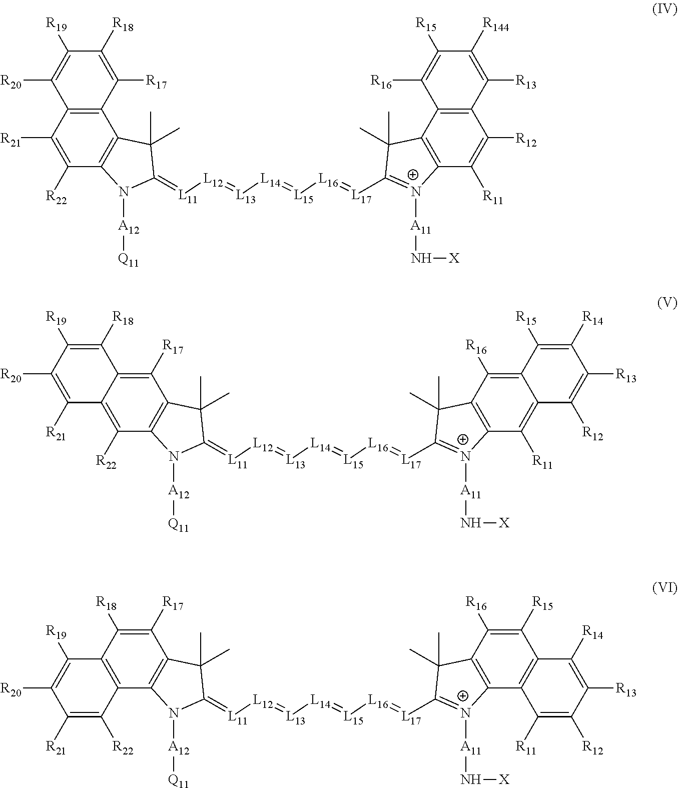

In formulas (IV) to (VI), R₁₁ to R₂₂ are the same as or different from each other and each represent a chemical species selected from the group consisting of a hydrogen atom, alkyl groups having a carbon number of 1 to 3, and halogen atoms.

L₁₁ to L₁₇ are the same as or different from each other and each represent a methine group that may be substituted by a methyl group or a halogen atom. A₁₁ and A₁₂ are the same as or different from each other and each represent an alkylene group having a carbon number of 1 to 10. Q₁₁ represents one selected from the group consisting of —OH, —CO₂H, —S(═O)₂OH, —P(═O)(OH)₂, and —OP(═O)(OH)₂, and X represents a structure formed by removing an amino group from an albumin.

Further features of the present disclosure will become apparent from the following description of exemplary embodiments with reference to the attached drawing.

### BRIEF DESCRIPTION OF THE DRAWINGS

The FIGURE is a plot showing the relationship between the pH of the aqueous solution separated by using ethyl acetate and the absorbance of the ethyl acetate phase separated from the aqueous solution.

### DESCRIPTION OF THE EMBODIMENTS

Exemplary embodiments of the subject matter of the present disclosure will now be described, but it is not limited to the disclosed embodiments.

The method for producing an optical imaging contrast agent according to an embodiment (hereinafter referred to as the method in some cases) includes at least the following steps.

### (a) Reaction Step

In this step, an albumin is reacted with a specific organic dye or a salt thereof in an aqueous solution to prepare an aqueous solution containing a combined form of the organic dye and the albumin.

### (b) pH Adjusting Step

In this step, the pH of the aqueous solution prepared in step (α) containing the combined form is adjusted to 7.2 or less.

### (c) Phase Separation Step

In this step, an ester is added to the aqueous solution adjusted to a pH of 7.2 or less in step (b), followed by mixing the mixture, and thus a liquid is prepared in which an organic solvent phase containing the ester and an aqueous solution phase are separated.

### (d) Extraction Step

In this step, an aqueous solution containing the combined form is extracted from the liquid prepared in step (c) in which the organic solvent phase and the aqueous solution phase are separated. The method of the present embodiment features converting the organic dye to be combined with the albumin into a hydrophilic structure that is unlikely to form an aggregate in an aqueous solution and including step (b).

As will be described later, a cyanine dye (typically having a structure in which two indole rings are linked with a methine chain) is used as the organic dye, and this organic dye is made unlikely to form an aggregate in an aqueous solution by binding a group having a hydrophilic functional group to the nitrogen of one of the indole rings. To the nitrogen of the other indole ring is bound a group (R₁₀₀ described later) that has a group easy to bind to the albumin and that becomes hydrophobic in a solution having a low pH. By converting the organic dye into such a structure, the organic dye is made easy to bind to the albumin. Consequently, the organic dye is unlikely to remain as it is without reacting with the albumin. Albumin changes reversibly in conformation depending on pH. If the albumin is conformationally changed in a low-pH aqueous solution, the organic dye adsorbed to the albumin is easy to separate from the albumin. In addition, the organic dye becomes more hydrophobic by the pH adjustment in step (b) and is, accordingly, likely to migrate to the organic solvent phase containing the ester in the phase separation in step (c). In other words, the organic dye itself is unlikely to be present in the aqueous solution phase. Therefore, the solution obtained in the extraction step does not much contain the organic dye itself, and a combined form accounts for a large proportion in the solution.

The steps of the method will now be described in detail.

### (a) Reaction Step

### Organic Dye

The organic dye used in this step is represented by any one of the following formulas (I) to (III):

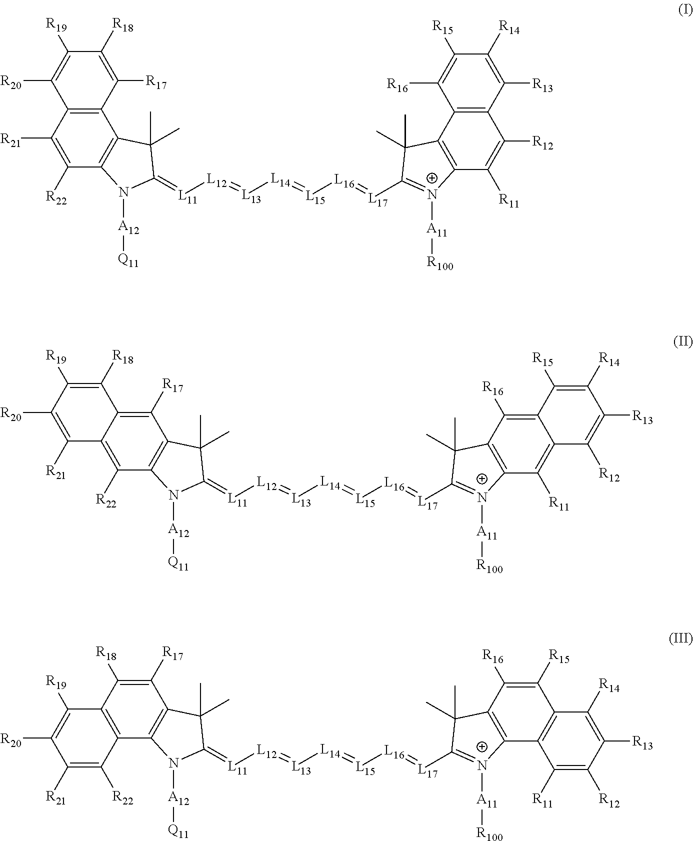

In formulas (I) to (III), R₁₁ to R₂₂ are the same as or different from each other and each represent a chemical species selected from the group consisting of a hydrogen atom, alkyl groups having a carbon number of 1 to 3, and halogen atoms. Desirably, R₁₁ to R₂₂ are each a hydrogen atom.

In formulas (I) to (III), L₁₁ to L₁₇ are the same as or different from each other and each represent a methine group that may be substituted by a hydrophobic group or an atom such as a methyl group or a halogen atom. When the methine group is substituted by a methyl group or a halogen atom, the organic dye is more hydrophobic in a state where it is easy to disperse in water. Accordingly, such an organic dye adsorbs easily the albumin and is thus likely to migrate to the organic solvent phase. Desirably, L₁₁ to L₁₇ are each an unsubstituted methine.

In formulas (I) to (III), A₁₁ and A₁₂ are the same as or different from each other and each represent an alkylene group having a carbon number of 1 to 10. Desirably, A₁₁ and A₁₂ are hydrophobic to the extent that they are easy to access the albumin, and are each an alkylene group having a carbon number of 2 to 5. Such an alkylene group is dispersible in water. More desirably, A₁₁ is an alkylene group having a carbon number of 5 and A₁₂ is an alkylene group having a carbon number of 4.

In formulas (I) to (III), Q₁₁ represents one selected from the group consisting of —OH, —CO₂H, —S(═O)₂OH, —P(═O)(OH)₂, and —OP(═O)(OH)₂. From the viewpoint of forming a structure unlikely to form an aggregate, Q₁₁ is desirably —S(═O)₂OH, which can impart hydrophilicity to compounds.

In formulas (I) to (III), R₁₀₀ represents a group represented by any one of the following formulas (i) to (viii).

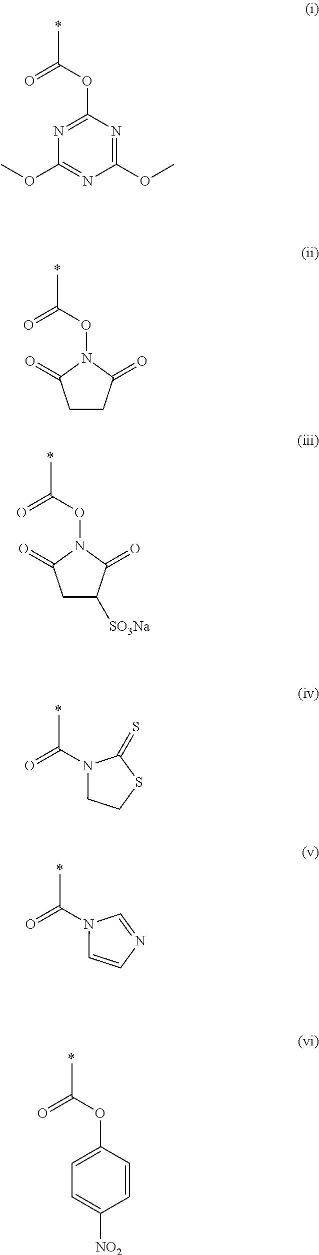

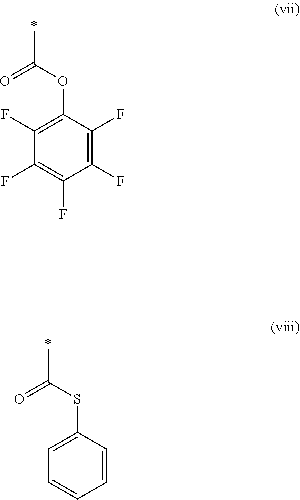

The asterisks * in formulas (i) to (viii) each indicate a bonding hand that will form a linkage with A₁₁ in formulas (I) to (III). The groups represented by formulas (i) to (viii) are reactive with an amino group, while it changes into a carboxyl group when being present in water for a long time. While the carboxyl group in a high-pH environment has a high probability of being in the form of COO⁻, the carboxyl group in a low-pH environment (in an acid condition) has a high probability of being in the form of COOH. Hence, as the pH is reduced, a larger portion of the organic dye changes into a form having the hydrophobic COOH and, accordingly, becomes likely to migrate to the hydrophobic solvent.

Also, the structures represented by formulas (I) to (III) have naphthyl rings, which have one more ring than the benzene rings of cyanine dyes such as Cy5 (registered trademark) and Cy7 (registered trademark), and are more hydrophobic. The structures of formulas (I) to (III) are therefore more adsorbable to albumin.

Benzo[e]indole as represented by formula (I), benzo[f]indole as represented by formula (II), and benzo[g]indole as represented by formula (III) have substantially the same hydrophobicity, tendency to aggregate, and adsorbability to albumin as each other.

The organic dye used in the present embodiment may absorb light in the range of near-infrared wavelengths to emit acoustic waves. The light in the range of near-infrared wavelengths has a wavelength in the range of 600 nm to 1300 nm.

The organic dye suitably used in the present embodiment may be represented by any one of the following formulas (I-1) to (I-4).

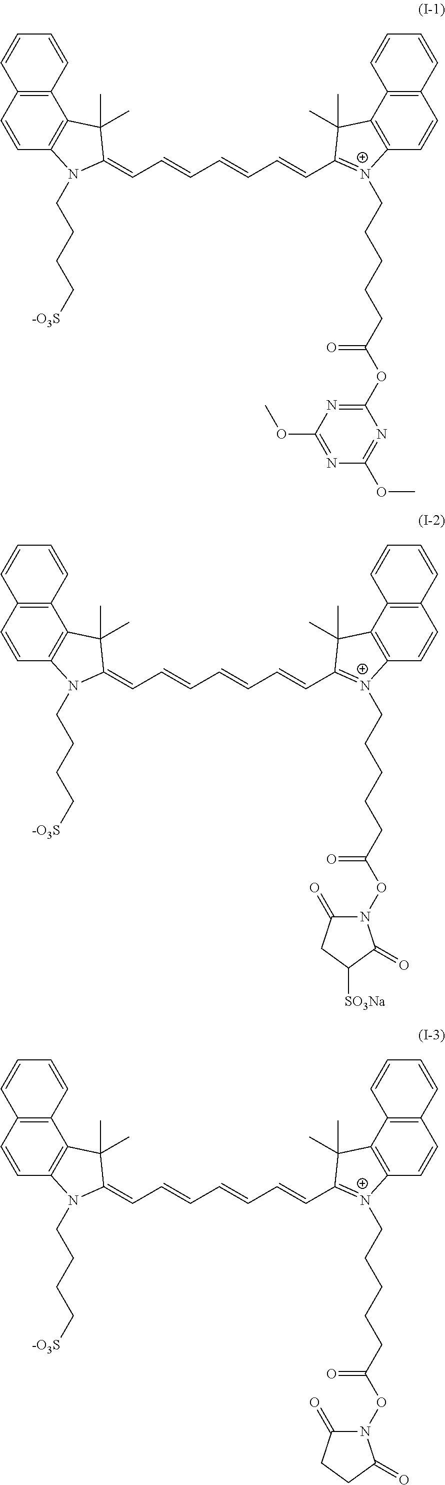

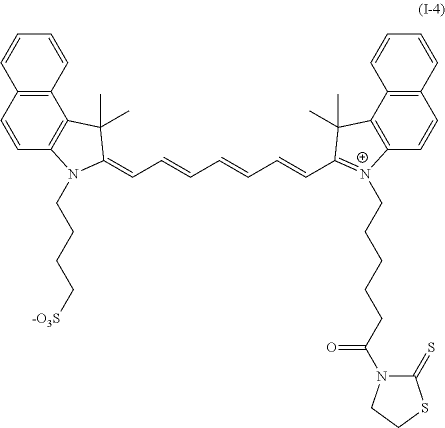

Alternatively, an organic dye represented by the following formula (I-5) may be activated with N-hydroxysuccinimide (NHS) or the like so as to be able to form easily an amide bond with an amino group, like the organic dyes of formulas (I-1) to (I-4).

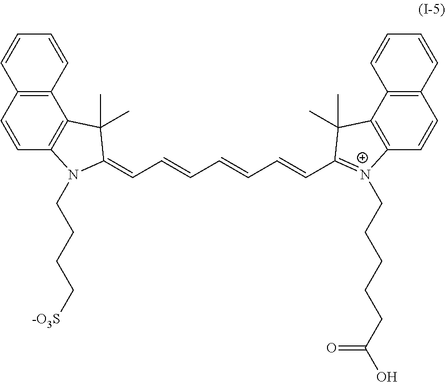

In an embodiment, a salt of the organic dye may be used as an alternative to the organic dye. In this instance, the cation of the salt may be sodium ion or potassium ion. Desirably, the salt is pharmaceutically acceptable. Albumin

The albumin used in the present embodiment is present in blood with a high content (35 g/L to 50 g/L), and is a protein having a molecular size of 66.5 kDa and made up of 585 amino acids. Albumin locally present in the living body has many functions, such as controlling osmotic pressure. Examples of the albumin used in the present embodiment include human serum albumin (hereinafter abbreviated as HSA) and bovine serum albumin (BSA). However, the albumin is not limited to these, and a modified HSA or BSA or a fragment of HSA or BSA may be used. Desirably, the albumin used in the present embodiment is selected from among HSA, a modified HSA, a fragment of HSA, and a fragment of a modified HSA. These are considered to be compatible with the human body. The albumin used in the present embodiment may be extracted from human blood or produced from Escherichia coli or the like. The albumin used in the present embodiment has a homology of 95% or more to the entire amino acid sequence of HSA or a partial sequence extracted from the entire sequence.

Albumin has a plurality of lysine residues or a free cysteine residue at positions the organic dye can access. One of the exemplary chemical bonds between albumin and the above-described organic dye is the amide bond formed with the amino group of a lysine residue of the albumin and the carboxyl group of the organic dye.

Reaction between Albumin and Organic Dye

The combined form of the present embodiment is formed by a coupling reaction of the albumin and the organic dye using their respective functional groups. For example, the albumin is combined with the organic dye by using the substituent on the nitrogen of one of the indole rings of the organic dye. It is desirable that the amino group of the albumin and the carboxyl group of the organic dye form an amide bond. For combining an organic dye with an albumin with an amide bond therebetween, in general, from the view point of increasing the reaction efficiency, an organic dye having any one of the groups represented by formulas (i) to (viii) is used. If these groups react with an amino group, an amide bond is formed. If these groups are present in water for a long time without reacting, they are converted into a carboxyl group. Since the amide bond is strong, it can keep stable in the living body.

The reaction between the organic dye and the albumin may be made by condensation using a condensing agent for a reaction between the carboxyl group and the amino group, by condensation through forming a salt for dehydration, or by using a dehydrator. Alternatively, the carboxyl group may be converted into one of the groups represented by formulas (i) to (viii), and this group is reacted with the amino group.

Examples of the condensing agent include carbodiimide-based condensing agents, imidazole-based condensing agents, triazine-based condensing agents, uronium-based condensing agents, and phosphoric acid-based condensing agents.

One of the examples of the triazine-based condensing agent may be 4-(4,6-dimethoxy-1,3,5-triazine-2-yl)-4-methylmorpholinium chloride (hereinafter abbreviated as DMT-MM) represented by the following formula (3):

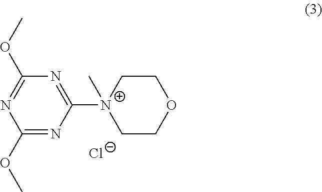 [SMILES: COc1nc(OC)nc([N+]2(C)CCOCC2)n1 ; [Cl-]]

DMT-MM is suitable as a condensing agent for combining a protein such as albumin with a cyanine-based compound. This is because DMT-MM allows condensation in water; hence it induces a condensation of a protein that is suitably present in an aqueous medium with the organic dye in an aqueous solution.

The moles of the condensing agent used are desirably more than or equal to the moles of the organic dye. In this instance, a larger amount of organic dye is combined with one molecule of the albumin. The condensing agent may be used as the reaction solvent.

The moles of the organic dye to be used for forming the combined form in the present embodiment are desirably more than or equal to the moles of the albumin. The more the organic dye is used, the more the organic dye is combined with one molecule of the albumin.

The aqueous solution used for the reaction in the present embodiment is not particularly limited as long as the organic dye and the albumin can be combined together. Examples of the aqueous solution include inorganic salt buffers, such as phosphate buffers, phosphate buffered saline, carbonate buffers, and borate buffers, Tris buffers, and Good buffers, such as MES buffer, MOPS buffer, HEPES buffer, tricine buffer, and bicine buffer. A mixture of two or more of these buffers may be used.

The pH of the aqueous solution for forming the combined form may be in, but is not limited to, the range of 4 to 10, and desirably in the range of 7 to 10. When the aqueous solution has a pH in the range of 7 to 10, the amino groups of the lysine of the albumin are more reactive, and, accordingly, the reaction efficiency between the organic dye and the albumin increases. The amount of the aqueous solution used for producing the compound may be appropriately set according to the reaction conditions. The reaction temperature in the reaction for forming the combined form may be in, but is not limited to, 0° C. to 60° C. It is advantageous to perform the reaction at a temperature suitable for the albumin. The reaction time may be, for example, in the range of 1 hour to 120 hours.

### (b) pH Adjusting Step

In this step, the pH of the aqueous solution containing the combined form prepared in the above-described reaction step is adjusted to 7.2 or less so that the organic dye can migrate easily to the organic solvent phase from the aqueous solution phase, and it is not particularly limited how the pH is adjusted. Advantageously, the pH of the aqueous solution containing the combined form is adjusted to 6.2 or less, desirably to 5.8 or less, more desirably to 4.8 or less. Since the acid dissociation constant of alkylcarboxyl groups is generally 4.8, a larger number of carboxyl groups of the organic dye molecules change from COO to COOH as the pH becomes close to 4.8 from 7.2. Accordingly, the organic dye becomes more hydrophobic and a large portion of the organic dye is likely to migrate to the organic solvent phase. For reducing the pH of the aqueous solution (or for making the solution acid), at least one of an acid aqueous solution and an acid solid salt may be added to the aqueous solution prepared in step (α). The acid aqueous solution may be a solution of hydrochloric acid, acetic acid, citric acid, or ammonium chloride. Acid solid salts include citric acid and ammonium chloride. A mixture of two or more of these solutions or solids may be used. A weak acid solution prepared by adding an alkaline aqueous solution, such as a sodium hydroxide solution, a sodium hydrogencarbonate solution, or a carbonate buffer, to a strong acid solution, such as a hydrochloric acid solution, may be used.

### (c) Phase Separation Step

In this step, an ester is added to the aqueous solution adjusted to a pH of 7.2 or less in step (b), and the mixture is stirred to prepare a liquid in which an organic solvent phase containing the ester and an aqueous solution phase are separated from each other.

### Ester

The ester used in the present embodiment is such that it enables the aqueous solution phase to be separated in the extraction and has affinity for the hydrophobic form of the organic dye. The ester may be a fatty acid ester. Examples of the fatty acid ester include ethyl acetate, propyl acetate, isopropyl acetate, butyl acetate, sec-butyl acetate, tert-butyl acetate, amyl acetate, ethyl propionate, and ethyl butyrate. A mixture of these organic solvents may be used. Acetic acid esters, such as ethyl acetate and butyl acetate, are more suitable. These organic solvents are polar and allow the organic dye to dissolve therein.

### (d) Extraction Step

In this step, an aqueous solution containing the combined form is extracted from the phase-separated liquid in which the organic solvent phase and the aqueous solution phase (or water phase) are separated from each other. The organic dye not combined with the albumin is extracted by using the organic solvent from the liquid prepared in step (c) while the combined form is kept in the aqueous solution phase. Then, the aqueous solution phase is collected to obtain the combined form.

In an embodiment, the extraction step may include drying the aqueous solution containing the combined form (reducing water). The drying may be performed by, but not limited to, a conventional method using, for example, an evaporator, a vacuum dryer, or a freeze dryer.

### Optical Imaging Contrast Agent

The optical imaging contrast agent of the present embodiment contains a combined form or a salt thereof that is made up of an albumin and at least one organic dye represented by any one of the above-described formulas (I) to (III) with a covalent bond therebetween.

The optical imaging contrast agent according to another embodiment contains an albumin, and a combined form or a salt thereof that is made up of an albumin and at least one organic dye with a covalent bond therebetween. The combined form is represented by any one of the following formulas (IV) to (VI).

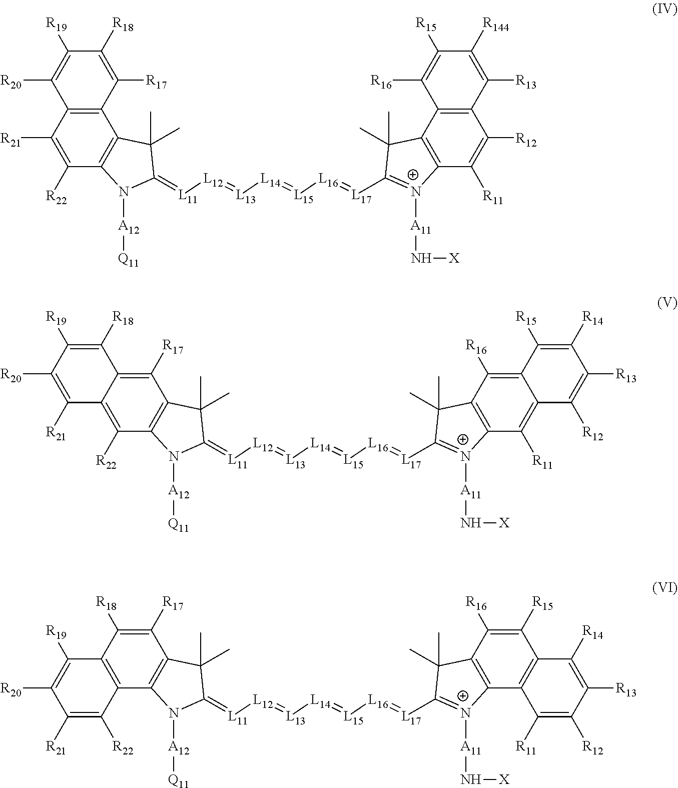

In formulas (IV) to (VI), R₁₁ to R₂₂ are the same as or different from each other and each represent a chemical species selected from the group consisting of a hydrogen atom, alkyl groups having a carbon number of 1 to 3, and halogen atoms.

In formulas (IV) to (VI), L₁₁ to L₁₇ are the same as or different from each other and each represent a methine group that may be substituted by a methyl group or a halogen atom.

In formulas (IV) to (VI), A₁₁ and A₁₂ are the same as or different from each other and each represent an alkylene group having a carbon number of 1 to 10.

In formulas (IV) to (VI), Q₁₁ represents one selected from the group consisting of —OH, —CO₂H, —S(═O)₂OH, —P(═O)(OH)₂, and —OP(═O)(OH)₂.

In formula (IV) to (VI), X represents a structure formed by removing an amino group from an albumin.

In an embodiment, the percentage of the moles of the combined form (the form of the organic dye combined with the albumin) to the total moles of all forms of the organic dye may be 60% or more and is advantageously 70% or more. When the form of the organic dye combined with the albumin accounts for a large proportion, the total amount of the organic dye to be administered to the subject can be reduced, and the percentage of the organic dye that can reach the tumor to the total moles of the organic dye that has been administered to the subject is increased. In addition, if the form of the organic dye not combined with the albumin remains in blood, signals can be received from portions other than the tumor. In view of this, the moles of the form of the organic dye combined with the albumin are 1.5 times or more those of the form of the organic dye not combined with the albumin. When the percentage of the moles of the combined form (or the organic dye combined with the albumin) to total moles of all forms of the organic dye is 60% or more, signals from the tumor are at least 1.5 times as strong as those from other portions even if all forms of the organic dye are evenly distributed to the tissue with the same proportion. Thus, the optical imaging contrast agent emits strong fluorescent or photoacoustic signals from the tumor.

In other words, the percentage of the moles of the uncombined form of the organic dye, or the organic dye not combined with the albumin, to the total moles of all forms of the organic dye in the aqueous solution obtained in the extraction step may be less than 40% and is advantageously less than 30%.

The optical imaging contrast agent of the present embodiment may further contain a dispersion medium in addition to the combined form. The dispersion medium used in the present embodiment is a liquid in which the combined form will be dispersed, and examples thereof include physiological saline, distilled water for injection, phosphate buffered saline, Ringer's solution, or glucose aqueous solution. The optical imaging contrast of the present embodiment may be in the form of a dispersion in which the combined form is dispersed in the dispersion in advance, or in the form of a kit including the combined form and the dispersion medium. In the case of the kit, the combined form is dispersed in the dispersion medium before administered to the living body. The optical imaging contrast agent may further contain a pharmacologically acceptable excipient and other additives, such as a vasodilator, a pH adjusting agent, a tonicity agent, a stabilizer, and a solubilizing agent. Furthermore, the optical imaging contrast agent may contain an additive used for freeze drying. Examples of such an additive include glucose, lactose, mannitol, polyethylene glycol, glycine, sodium chloride, and sodium hydrogenphosphate. Additives may be used singly or in combination.

The solution of the optical imaging contrast agent may be adjusted to a pH in the range 5.0 to 8.0, desirably 7.0 to 7.4, when administered to the living body. The osmotic pressure of this solution is desirably 0.01 to 2.0, more desirably 1.0, with respect to that of physiological saline.

Optical imaging mentioned herein refers to imaging with light irradiation. More specifically, photoacoustic imaging is performed by detecting the acoustic waves emitted from the contrast agent by irradiating the contrast agent with light, and fluorescent imaging is performed by detecting fluorescence emitted from the contrast agent by irradiating the contrast agent with light. Photoacoustic imaging is a concept including photoacoustic tomography (sectional radiography). The optical imaging contrast agent of the present disclosure is called the fluorescent imaging contrast agent when it is used for fluorescent imaging, and is called the photoacoustic imaging contrast agent when it is used for photoacoustic imaging.

### Tumor Imaging

The optical imaging contrast agent of the present embodiment administered to a living body is accumulated in a tumor site by the enhanced permeability and retention (EPR) effect. Therefore, the tumor site of the living body can be imaged by irradiating the living body with light after the optical imaging contrast agent has been administered to the living body, and detecting the acoustic waves or fluorescence emitted from the contrast agent.

### (Lymph Node Contrast Agent)

The optical imaging contrast agent of the present embodiment may also be used for imaging lymph nodes. The optical imaging contrast agent is particularly suitable for imaging the sentinel lymph node. If the above-described organic dye is administered to a living body for imaging the sentinel lymph node, the observation period is limited because the organic dye is immediately transferred to blood and discharged from the body. However, the molecule of the optical imaging contrast agent of the present embodiment is larger in size than the organic dye itself. Accordingly, the dispersion speed of the optical imaging contrast agent to the tissue decreases, and it is expected that the residence time of the contrast agent in the sentinel lymph node becomes longer. Thus, the optical imaging contrast agent of the present embodiment can be suitably used for imaging the sentinel lymph node.

### Capture Molecule

A capture molecule may be bound to the combined form used in the present embodiment. The capture molecule mentioned herein is a substance capable of specifically binding to a target site such as a tumor or a substance capable of binding to a substance present around the target site, and can be arbitrarily selected from among biomolecules and chemical substances such as medicines. Examples of the capture molecule include antibodies, antibody fragments, artificial antibodies such as single chain antibodies, enzymes, bioactive peptides, glycopeptides, carbohydrates, lipid, and molecular recognition compounds. These capture molecules may be used singly or in combination. The use of the combined form to which such a capture molecule is bound enables the specific detection of a target site and the trace of the dynamics, localization, drug efficacy, metabolism, and the like of a target substance.

### Optical Imaging Method Using the Contrast Agent

A method will now be described for detecting the optical imaging contrast agent administered to a living body with a photoacoustic imaging apparatus or a fluorescent imaging apparatus. The method for detecting the optical imaging contrast agent of the present embodiment includes the following process steps (α) and (β) and may optionally include other steps: (α) irradiating a subject to which the optical imaging contrast agent has been administered with a light ray having a wavelength in the range of 600 nm to 1300 nm; and (β) detecting the acoustic waves or fluorescence emitted from the optical imaging contrast agent in the subject.

This method may further include the step of recreating spatial photoacoustic signal intensity distribution or fluorescent signal intensity distribution from the information obtained in step (β) including the wavelength, phase and time of the acoustic waves or fluorescence. A three-dimensional image reconstruction may be performed based on the information obtained in step (β) including the wavelength, phase and time of the photoacoustic signals or fluorescence. The data obtained by the image reconstruction may be in any form as long as the positional information of the intensity distribution of the photoacoustic signals or fluorescence can be known. For example, the data may be in a form that represents photoacoustic signal intensity or fluorescence intensity in a three-dimensional space or in a form that represents photoacoustic signal intensity or fluorescence intensity in a two-dimensional plane. The information may be obtained by imaging an identical observation target by different methods. Thus, the positional correspondence between the information and the intensity distribution of the photoacoustic signals or fluorescence can be obtained.

In step (α), the optical imaging contrast agent may be administered to the subject by oral administration, injection, or the like.

The apparatus or light source for irradiating the subject in step (α) and the apparatus for detecting the photoacoustic signals or fluorescence emitted from the optical imaging contrast agent in step (β) are not particularly limited.

The light source used in step (α) is such that it can irradiate the subject with pulsed laser light having a wavelength in the range of 600 nm to 1300 nm, and is not otherwise limited. Examples of the apparatus that emits pulsed laser light include a titanium-sapphire laser (LT-2211-PC, manufactured by Lotis), an OPO laser (LT-2214 OPO, manufactured by Lotis), and an Alexandrite laser.

For detecting acoustic waves, various apparatuses may be used without particular limitation. For example, a commercially available photoacoustic imaging apparatus Nexus 128 (manufactured by Endra Inc.) may be used.

For detecting fluorescence, various apparatuses may be used without particular limitation. For example, a commercially available fluorescent imaging apparatus IVIS Imaging System (manufactured by PerkinElmer Inc.) may be used.

The imaging method using the optical imaging contrast agent of the present embodiment, which includes the above-described steps (α) and (β), enables a target site, such as a tumor, a lymph node, or a blood vessel, to be imaged.

### EXAMPLES

Although the subject matter of the present disclosure will be further described with reference to Examples, various modifications to material, composition, reaction conditions, and the like may be made effectively without being limited to the following Examples.

### Analysis

In the following Examples, absorption spectra were measured with an ultraviolet-visible absorption spectrum measuring apparatus Gene Quant (manufactured by GE Healthcare).

pH was measured with a compact pH meter Twin pH (manufactured by HORIBA).

### Example 1

### Preparation of Reaction Liquid Containing Organic Dye and Albumin

An albumin aqueous solution was prepared by dissolving 400 mg (6.0 μmol) of human serum albumin (hereinafter abbreviated as HSA) in 62.5 mL of 50 mM carbonate buffer (pH 9.6). To 15 mL of chloroform were added 50 mg (68 μmol) of an organic dye represented by formula (I-5) and 95 mg of DMT-MM.nH₂O. The mixture was stirred at room temperature in a dark place for 2 hours. Thus, a solution containing the organic dye represented by formula (I-1) was prepared. After removing the solvent from the solution by using an evaporator, 13 mL of ethanol was added to the resulting liquid, followed by suction filtration. The filtrate was collected. The collected filtrate (11 mL) was added to the albumin aqueous solution (62.5 mL). The mixture was stirred at room temperature in a dark place for 3 hours to yield a reaction liquid.

### Measurement of Organic Dye Content in Liquid-Liquid Extracted Ethyl Acetate Phase

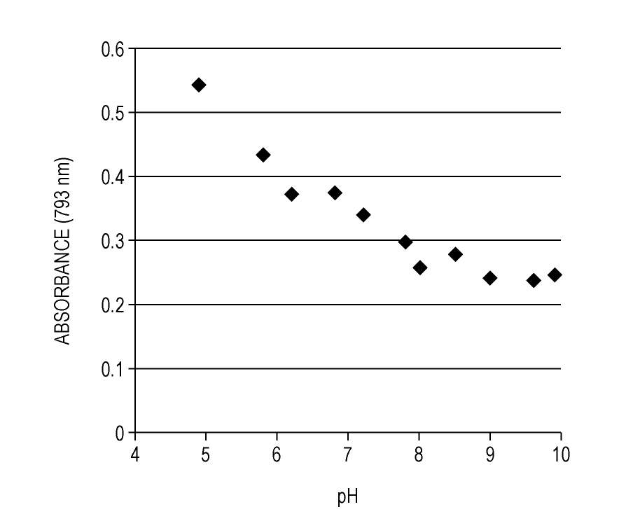

To 0.5 mL of the reaction liquid was added 0.5 mL of mixture of 1N HCl aqueous solution and 50 mM pH 9.6 carbonate buffer (HCl:carbonate buffer (CB)=1:8, 1:9, or 1:10) or 0.5 mL of a dilute of the mixture with distilled water. The pH of the resulting reaction liquids was measured. Then, the dilute was further added to the mixtures so that the resulting liquid each have a pH of 4.9, 5.8, 6.2, 6.8, 7.2, 7.8, 8.0, 8.5, 9.0, 9.6, or 9.9 (each 1.0 mL). To each of 11 reaction liquids having the respective pHs (each 1.0 mL), 4.0 mL of ethyl acetate was added, followed by shaking for agitation. After an organic solvent phase containing the ethyl acetate separated from the aqueous solution phase, the organic solvent phase or the ethyl acetate phase (4.0 mL) was collected. Hereinafter, the organic solvent phase containing the ethyl acetate is referred to as the ethyl acetate phase and the aqueous solution phase is referred to as the water phase. Each of the 11 ethyl acetate phases thus collected (each 0.5 mL) was diluted with 0.5 mL of ethyl acetate for measuring the absorption spectrum. FIG. 1 shows the results of the absorbance of the ethyl acetate phase collected from each of the solutions having different pHs, measured at an absorption wavelength of 793 nm.

The results shown in FIG. 1 suggest that the organic dye in the reaction liquid having a pH of 7.2 or less is protonated and thus becomes more hydrophobic, and that a large portion of the organic dye not combined with the albumin in the reaction liquid is thus removed from the reaction liquid by liquid-liquid extraction using ethyl acetate.

### Example 2

HSA was dissolved in 50 mM carbonate buffer (pH 9.6) to prepare 10 mg/mL albumin aqueous solution. An organic dye solution was prepared by dissolving 1 mg of an organic dye represented by formula (I-2), ICG-Sulfo-OSu (registered trademark, produced by Dojindo Laboratories), in 0.1 mL of DMSO. The resulting organic dye solution (0.04 mL) was added to 0.40 mL of the albumin aqueous solution, and the mixture was stirred at room temperature in a dark place for 3 hours. The reaction liquid thus prepared was acidified by adding 0.46 mL of mixture of 1N HCl aqueous solution and 50 mM pH 9.6 carbonate buffer (HCl:CB)=1:10) to 0.43 mL of the reaction liquid. To the resulting reaction liquid, 4.0 mL of ethyl acetate was added, followed by shaking for agitation. After the ethyl acetate phase separated from the water phase, the water phase was collected. A series of operations including adding another 4.0 mL of ethyl acetate to the collected water phase, shaking the mixture for agitation, and collecting the water phase was repeated twice. The collected water phase was replaced with 1×PBS buffer by ultrafiltration using 30K, and thus a solution containing a combined form of ICG-Sulfo-OSu and HSA with a covalent bond was prepared.

### Example 3

The reaction liquid prepared in the same manner as in Example 2 was acidified by adding 0.42 mL of 30 mM citric acid aqueous solution to 0.43 mL of the reaction liquid. To the resulting reaction liquid, 4.0 mL of ethyl acetate was added, followed by shaking for agitation. After the ethyl acetate phase separated from the water phase, the water phase was collected. A series of operations including adding another 4.0 mL of ethyl acetate to the collected water phase, shaking the mixture for agitation, and collecting the water phase was further repeated twice. The collected water phase was replaced with 1×PBS buffer by ultrafiltration using 30K, and thus a solution containing a combined form of ICG-Sulfo-OSu and HSA with a covalent bond was prepared.

### Example 4

The reaction liquid prepared in the same manner as in Example 2 was acidified by adding 0.42 mL of 10 mM citric acid aqueous solution to 0.43 mL of the reaction liquid. To the resulting reaction liquid, 4.0 mL of ethyl acetate was added, followed by shaking for agitation. After the ethyl acetate phase separated from the water phase, the water phase was collected. A series of operations including adding another 4.0 mL of ethyl acetate to the collected water phase, shaking the mixture for agitation, and collecting the water phase was further repeated twice. The collected water phase was replaced with 1×PBS buffer by ultrafiltration using 30K, and thus a solution containing a combined form of ICG-Sulfo-OSu and HSA with a covalent bond was prepared.

### Comparative Example 1

A solution containing a combined form of ICG-Sulfo-OSu and HSA with a covalent bond was prepared by ultrafiltration using 50K for replacement with 1×PBS buffer, but the pH of the reaction solution (0.43 mL) prepared in the same manner as in Example 2 was not adjusted. Determination of Organic Dye not Combined with Albumin

In Examples 2 to 4 and the Comparative Example, the percentage of ICG-Sulfo-OSu not combined with the albumin in the finally obtained solution containing the combined form was determined. More specifically, each of the solutions was subjected to SDS-PAGE (Sodium dodecyl sulfate-polyacrylamide gel electrophoresis), and the electrophoretic gel was measured with a fluorescence imager. First, the molarity of the organic dye in each solution was determined by measuring absorbance. For each gel lane, the solution containing the organic dye in an amount equivalent to 1.0 pmol of all forms of the organic dye and ICG-Sulfo-OSu solution in an amount equivalent to 0.125 pmol, 0.25 pmol, 0.5 pmol, or 1.0 pmol of ICG-Sulfo-OSu were added for electrophoresis. The fluorescence intensity of the low-molecular-weight band in each gel lane after electrophoresis was measured by using ODYSSEY (registered trademark) CLx Infrared Imaging System (produced by LI-COR). For each of the Examples and the Comparative Example, the moles of ICG-Sulfo-OSu not combined with the albumin in the electrophoretic gel was calculated from a calibration curve prepared by using fluorescence intensities of the lanes of the ICG-Sulfo-OSu solution. The Table shows the percentage of the ICG-Sulfo-OSu not combined with the albumin to 1.0 pmol of all forms of the organic dye added to the electrophoretic gel.

TABLE

| | | | Percentage | | Percentage | | |
|---|---|---|---|---|---|---|---|
| | | | (%) of organic | Percentage | (%) of organic | Percentage | |
| | | | dye not | (%) of organic | dye not | (%) of organic | Percentage |
| | | Acid solution | combined | dye combined | combined | dye combined | (%) of |
| | | for Liquid- | with albumin | with albumin | with albumin | with albumin | organic dye |
| | | liquid | after | after | before | before | reduced by |
| | Purification | extraction | purification | purification | purification | purification | purification |
| Example 2 | Liquid-liquid | Mixture of | 26 | 74 | 62 | 38 | 36 |
| | extraction and | hydrochloric | | | | | |
| | ultrafiltration | acid and | | | | | |
| | | carbonate | | | | | |
| | | buffer | | | | | |
| Example 3 | Liquid-liquid | 30 mM Citric | 36 | 64 | 75 | 25 | 39 |
| | extraction and | acid | | | | | |
| | ultrafiltration | | | | | | |
| Example 4 | Liquid-liquid | 10 mM Citric | 28 | 72 | 75 | 25 | 47 |
| | extraction and | acid | | | | | |
| | ultrafiltration | | | | | | |
| Comparative | Ultrafiltration | None | 61 | 39 | 31 | 31 | 8 |
| Example 1 | | | | | | | |

### CONCLUSION

The results shown in the Table suggest that the use of citric acid or a mixture of hydrochloric acid and a carbonate buffer as an acid solution to be added to the reaction liquid enables a large portion of the organic dye not combined with the albumin to be removed from the reaction liquid by liquid-liquid extraction.

In the method for producing an optical imaging contrast agent of the present disclosure, a specific organic dye is reacted with an albumin in an aqueous solution, then the pH of the resulting aqueous solution is reduced, and the aqueous solution with a low pH was subjected to phase separation using ethyl acetate. Thus, the amount of the organic dye remaining as it is in the finally prepared solution can be reduced.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-009306 filed Jan. 20, 2016, which is hereby incorporated by reference herein in its entirety.

What is claimed is:

1. A method for producing an optical imaging contrast agent, the method comprising:
reacting an albumin with a specific organic dye or a salt thereof in an aqueous solution to prepare an aqueous solution containing a combined form of the organic dye and the albumin with a covalent bond therebetween, the organic dye being represented by any one of the following formulas (I) to (III): 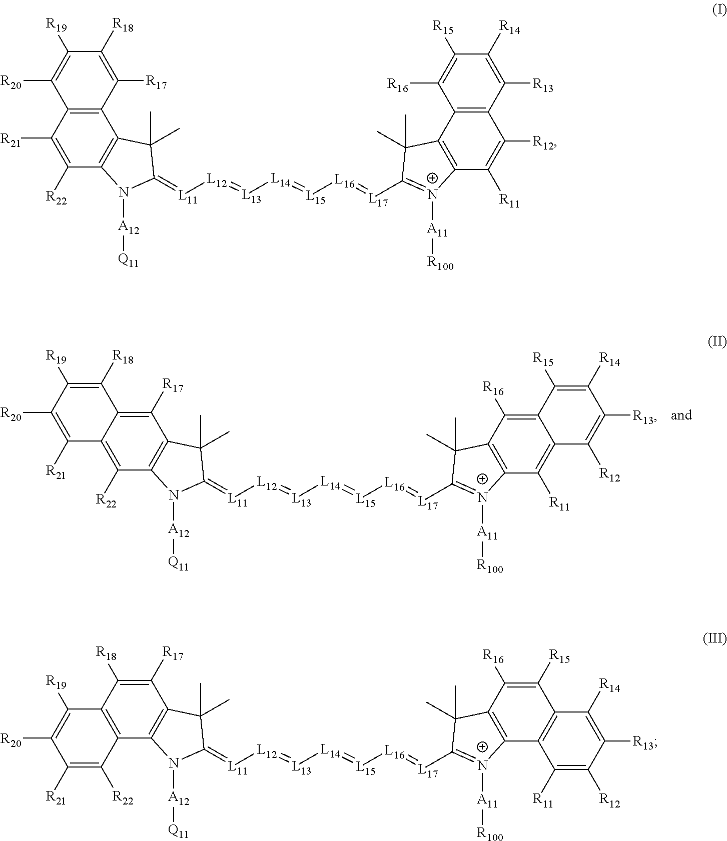
adjusting the pH of the aqueous solution containing the combined form to 7.2 or less;
adding an ester to the aqueous solution having a pH of 7.2 or less, thereby preparing a liquid in which an organic solvent phase containing the ester and an aqueous solution phase are separated from each other; and
extracting an aqueous solution containing the combined form from the phase-separated liquid,
wherein in formulas (I) to (III), R₁₁ to R₂₂ are the same as or different from each other and each represent a chemical species selected from the group consisting of a hydrogen atom, alkyl groups having a carbon number of 1 to 3, and halogen atoms, L₁₁ to L₁₇ are the same as or different from each other and each represent a methine group that may be substituted by a methyl group or a halogen atom, A₁₁ and A₁₂ are the same as or different from each other and each represent an alkylene group having a carbon number of 1 to 10, Q11 represents one selected from the group consisting of —OH, —CO₂H, —S(═O)₂OH, —P(═O)(OH)₂, and —OP(═O)(OH)₂, and R₁₀₀ represents a group represented by any one of the following formulas (i) to (viii): 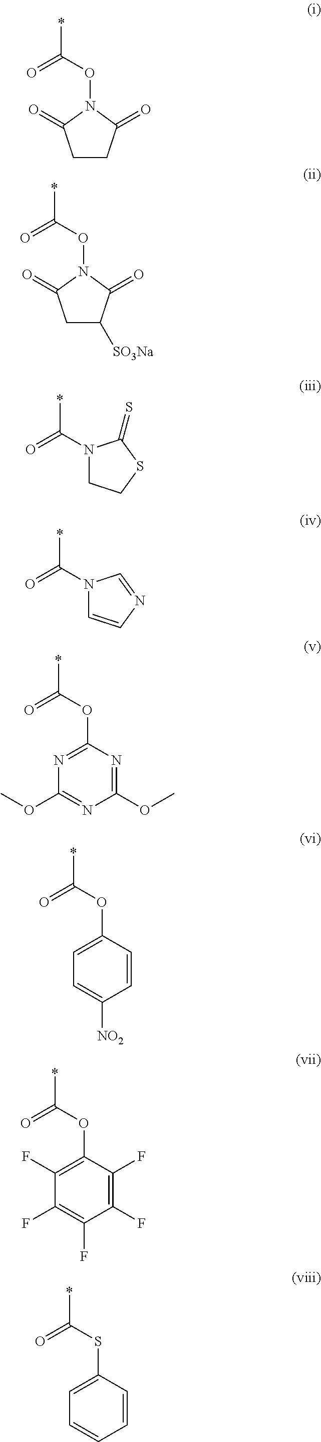
wherein * in formulas (i) to (viii) each indicate a bonding hand that will form a linkage with A₁₁ in formulas (I) to (III).

2. The method according to claim 1, wherein the pH of the aqueous solution containing the combined form is adjusted to 6.2 or less.

3. The method according to claim 1, wherein the pH of the aqueous solution containing the combined form is adjusted to 5.8 or less.

4. The method according to claim 1, wherein the adjusting of the pH includes adding hydrochloric acid or citric acid to the aqueous solution containing the combined form.

5. The method according to claim 1, wherein the ester is ethyl acetate.

6. The method according to claim 1, wherein R₁₁ to R₂₂ in formulas (I) to (III) each represent a hydrogen atom.

7. The method according to claim 1, wherein L₁₁ to L₁₇ in formulas (I) to (III) each represent an unsubstituted methine group.

8. The method according to claim 1, wherein A₁₁ and A₁₂ in formulas (I) to (III) each represent an alkylene group having a carbon number of 2 to 5.

9. The method according to claim 1, wherein Q₁₁ in formulas (I) to (III) represents —S(═O)₂OH.

10. The method according to claim 1, wherein the organic dye is represented by any one of the following formulas (I-1) to (I-4):

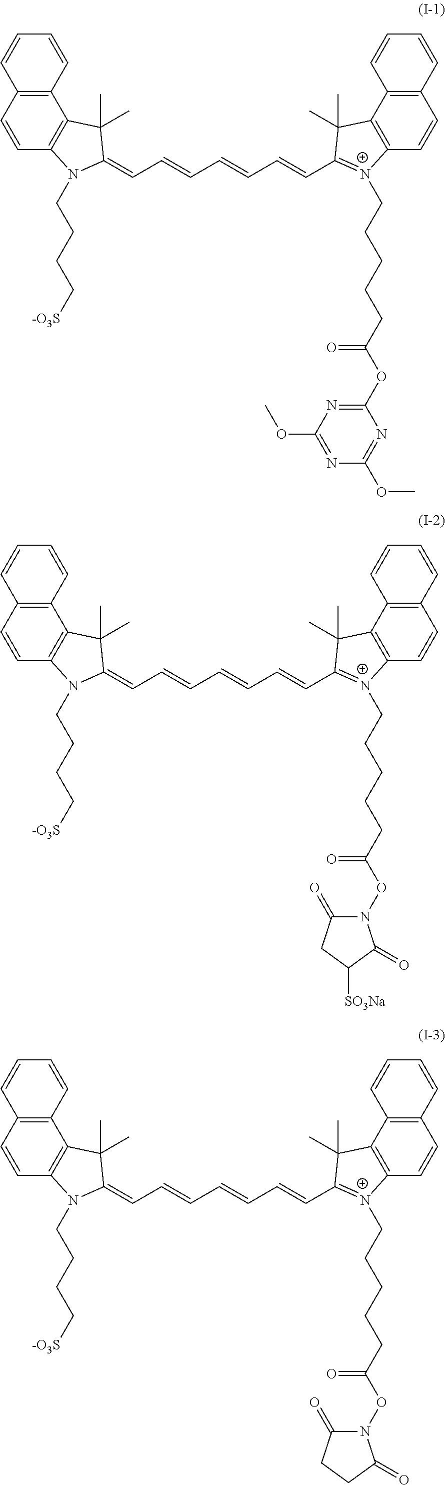

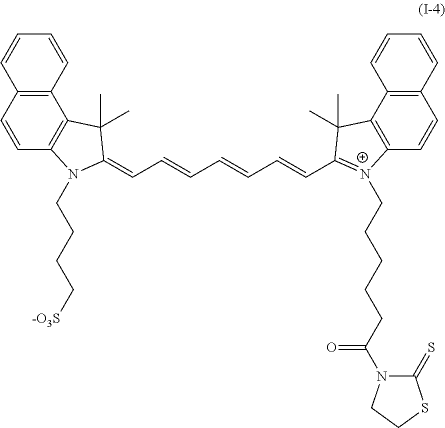

11. The method according to claim 1, wherein the covalent bond is an amide bond.

12. The method according to claim 1, wherein the albumin is one selected from the group consisting of human serum albumin, a modified human serum albumin, a fragment of human serum albumin, and a fragment of modified human serum albumin.

13. The method according to claim 1, wherein the form of the organic dye combined with the albumin accounts for 60% or more of the total moles of all forms of the organic dye in the aqueous solution obtained by the extraction.

14. The method according to claim 1, wherein the form of the organic dye combined with the albumin accounts for 70% or more of the total moles of all forms of the organic dye in the aqueous solution obtained by the extraction.

15. An optical imaging contrast agent comprising:
a combined form of an albumin and at least one organic dye or a salt thereof with a covalent bond therebetween, the form of the organic dye combined with the albumin accounting for 60% or more of the total moles of all forms of the organic dye in the optical imaging contrast agent, the organic dye represented by any one of the following formulas (I) to (III): 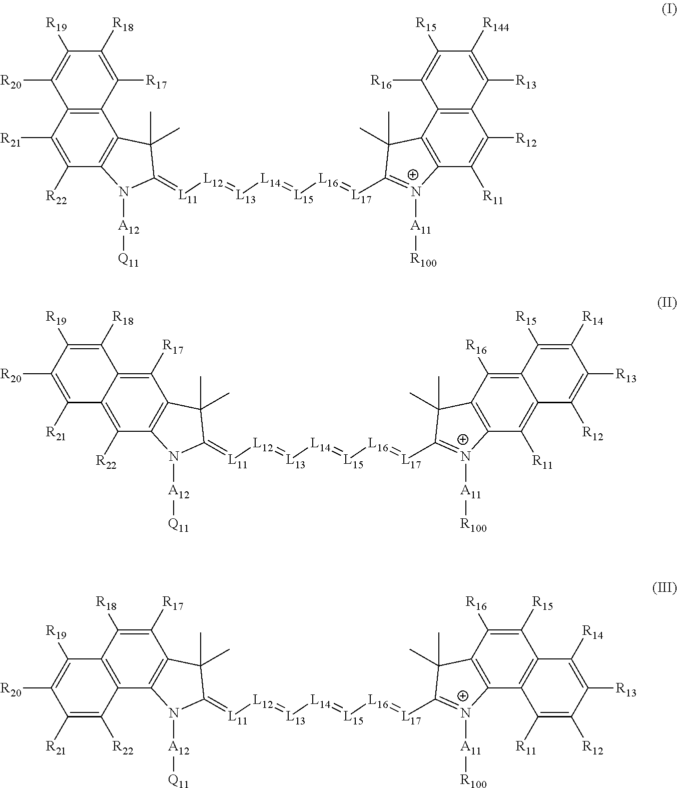
wherein in formulas (I) to (III), R₁₁ to R₂₂ are the same as or different from each other and each represent a chemical species selected from the group consisting of a hydrogen atom, alkyl groups having a carbon number of 1 to 3, and halogen atoms, L₁₁ to L₁₇ are the same as or different from each other and each represent a methine group that may be substituted by a methyl group or a halogen atom, A₁₁ and A₁₂ are the same as or different from each other and each represent an alkylene group having a carbon number of 1 to 10, Q₁₁ represents one selected from the group consisting of —OH, —CO₂H, —S(═O)₂OH, —P(═O)(OH)₂, and —OP(═O)(OH)₂, and R₁₀₀ represents a group represented by any one of the following formulas (i) to (viii): 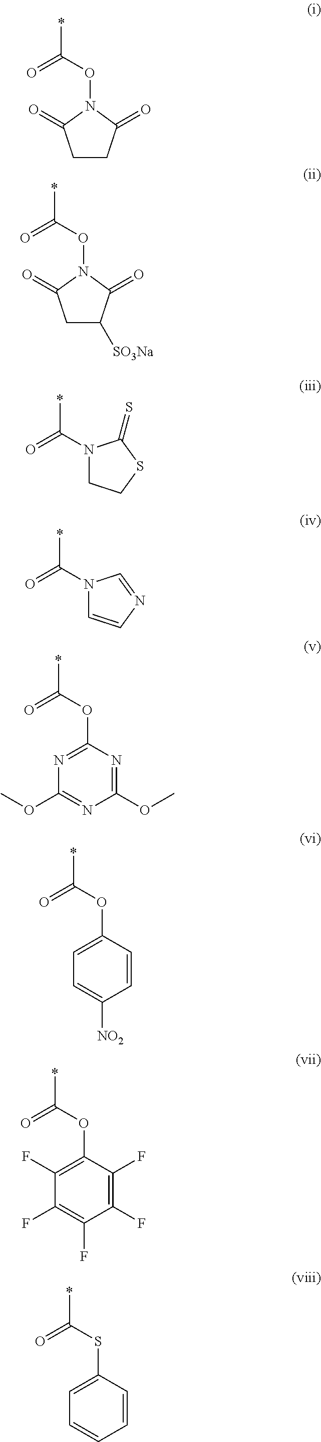
wherein * in formulas (i) to (viii) each indicate a bonding hand that will form a linkage with A₁₁ in formulas (I) to (III).

16. An optical imaging contrast agent comprising:
an albumin; and
a combined form or a salt thereof, the combined form being made up of an albumin and at least one organic dye with a covalent bond therebetween, the form of the organic dye being a portion of the combined form accounting for 60% or more of the total moles of all forms of the organic dye in the optical imaging contrast agent, the combined form being represented by any one of the following formulas (IV) to (VI): 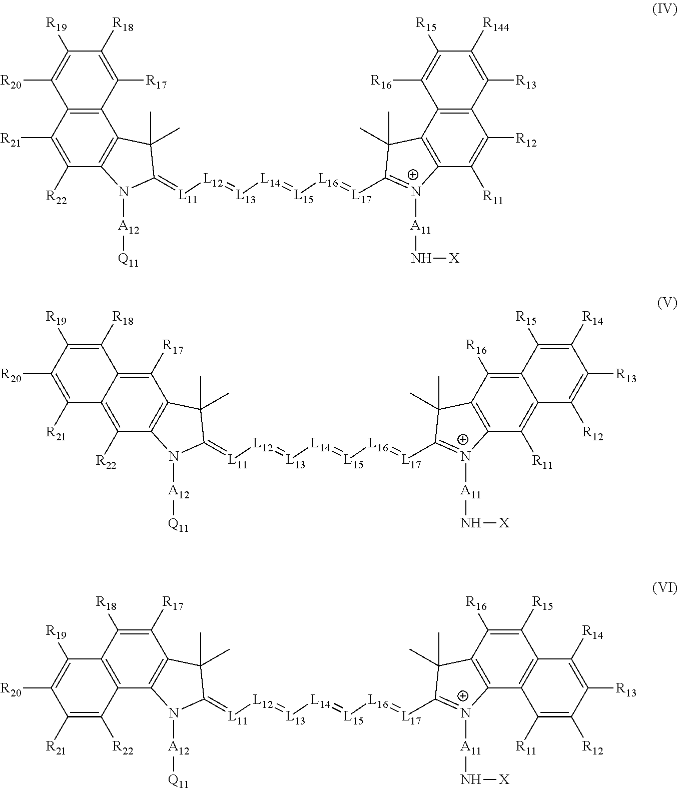
wherein in formulas (IV) to (VI), R₁₁ to R₂₂ are the same as or different from each other and each represent a chemical species selected from the group consisting of a hydrogen atom, alkyl groups having a carbon number of 1 to 3, and halogen atoms, L₁₁ to L₁₇ are the same as or different from each other and each represent a methine group that may be substituted by a methyl group or a halogen atom, A₁₁ and A₁₂ are the same as or different from each other and each represent an alkylene group having a carbon number of 1 to 10, Q₁₁ represents one selected from the group consisting of —OH, —CO₂H, —S(═O)₂OH, —P(═O)(OH)₂, and —OP(═O)(OH)₂, and
X represents a structure formed by removing an amino group from an albumin.

17. The optical imaging contrast agent according to claim 15, wherein the form of the organic dye being a portion of the combined form accounts for 70% or more of the total moles of all forms of the organic dye in the optical imaging contrast agent.

18. The optical imaging contrast agent according to claim 16, wherein R₁₁ to R₂₂ in formulas (IV) to (VI) each represent a hydrogen atom.

19. The optical imaging contrast agent according to claim 16, wherein L₁₁ to L₁₇ in formulas (IV) to (VI) each represent an unsubstituted methine group.

20. The optical imaging contrast agent according to claim 16, wherein A₁₁ and A₁₂ in formulas (IV) to (VI) each represent an alkylene group having a carbon number of 2 to 5.

21. The optical imaging contrast agent according to claim 16, wherein Q₁₁ in formulas (IV) to (VI) represents —S(═O)₂OH.

22. The optical imaging contrast agent according to claim 15, wherein the combined form is a compound represented by the following formula (IV-1) or a salt thereof: [chemical structure]
